(19) United States
(12) Patent Application Publication
(10) Pub. No.: US 2024/0224401 A1
(43) Pub. Date: Jul. 4, 2024

(54) System and Method for Providing an Immersive Experience

(71) Applicant: Nicholas Dimatteo, III, Flower Mound, TX (US)

(72) Inventor: Nicholas Dimatteo, III, Flower Mound, TX (US)

(21) Appl. No.: 18/403,716

(22) Filed: Jan. 3, 2024

Related U.S. Application Data
(60) Provisional application No. 63/436,836, filed on Jan. 3, 2023.

(51) Int. Cl. H05B 47/155; A61L 9/12; G06F 3/0482; G10H 1/36

(52) U.S. Cl. CPC H05B 47/155; A61L 9/125; G06F 3/0482; G10H 1/361

(57) ABSTRACT

A system and method for an immersive light experience. The physical components are installed in an enclosed room, an open room/bar area, entryway, game rooms, restaurant, restaurant sections, customer gathering areas, or prize redemption areas in an arcade, casino, Location Based Entertainment (LBE), Family Entertainment Centers (FEC), or similar establishments and are generally located across a ceiling. A scent dispenser is located on the ceiling as well. A control panel can be located or mounted on a wall or at any other location as desired. A control panel can be comprised of a selectable menu via a touch panel for selecting pre-programmed immersive experience programs as desired for the immersive experience desired to be delivered. A typical on/off button could be used as a remote control panel or button to simply turn on and off the experience, or to start a pre-programmed experience for execution/delivery to the room.

### SEQUENCE LISTING OR PROGRAM

Not Applicable

### TECHNICAL FIELD OF THE INVENTION

The present invention relates generally to an audio visual show. More specifically, the present invention relates an immersive experience providing one or more people an immersive audio and visual experience.

### BACKGROUND OF THE INVENTION

In a standard audio visual experience, participants typically sit in seats and watch a show appear on a stage on a fixed point in front of them typically stimulating only sight and sound. An immersive experience tends to stimulate all five senses—sight and sound, as with conventional displays, but also touch, and frequently taste and even smell.

An immersive experience makes individual participants feel as if they have had a uniquely personal experience, that they are not just part of the crowd. At the same time, immersive experiences often emphasize social interactions, either through directed tasks in small groups, or by fostering a looser party atmosphere.

An immersive experience describes the perception of being surrounded by—and being a part of—a different environment than our normal day to day. This could be facilitated with technology like Virtual Reality (VR) goggles or a physical environment like a theme park attraction with multi-media components.

The typical way to have an immersive experience is in a large theater gathering, using expensive VR equipment, and/or paying an expensive admission to a theme park, ride, or similar attraction, which is not easily available for quick and easy reuse in an on-demand capacity in a location.

Therefore, what is needed is a system and method for providing an immersive experience in an on-demand manner to a group of people that does not require expensive tech equipment that can be damaged or needs to be purchased in large quantities or large, fixed locations.

A system and method for equipping a location or space for an on-demand immersive experience for a number of themes or applications is needed in the market.

### SUMMARY OF THE INVENTION

The present invention is a system and method for an immersive light experience. The physical components show can be installed in an enclosed room, an open room/bar area, entryway, game rooms, restaurant, restaurant sections, customer gathering areas, or prize redemption areas in an arcade, casino, Location Based Entertainment (LBE), Family Entertainment Centers (FEC), or similar establishments.

The physical components, in one such embodiment, are a plurality of lights and speakers are generally located across a ceiling. Additionally, a scent dispense can be located on a ceiling as well. A control panel can be located or mounted on a wall or at any other location as desired.

A control panel can be comprised of a selectable menu via a touch panel for selecting pre-programmed immersive experience programs such as “Happy Birthday”, “Color Lights”, and “Sports Club Song”, and image mapping as desired for the immersive experience desired to be delivered.

A typical on/off button could be used as a remote control panel or button to simply turn on and off the experience, or to start a pre-programmed experience for execution/delivery to the room. In another embodiment the button could be a multi-button with colored buttons for selecting different pre-programmed themes or immersive experience routines.

In one embodiment a microprocessor and controller are connected between the control panel and physical components such as the lights, speakers, scent dispenser, etc. for the storing in memory one or more pre-programmed immersive experiences and for controlling, presenting, and delivering a selected pre-programmed show to the room and participants for a fixed period of time.

### BRIEF DESCRIPTION OF THE DRAWINGS

The accompanying drawings, which are incorporated herein a form a part of the specification, illustrate the present invention and, together with the description, further serve to explain the principles of the invention and to enable a person skilled in the pertinent art to make and use the invention.

FIG. 1 is a representation of the physical layout and components of the present invention in one exemplary embodiment.

FIG. 2a is a representation of the physical control panel in one exemplary embodiment.

FIG. 2b is a representation of the physical control panel in another exemplary embodiment.

FIG. 3 is a flow chart illustrating the physical components executing and controlling the audio and visual components of the immersive experience.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

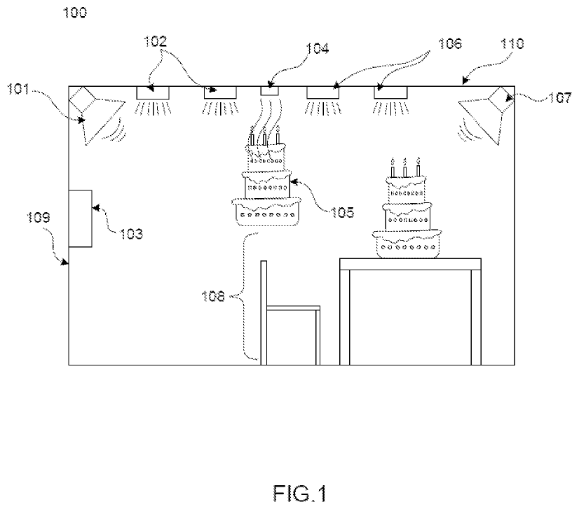

Now referring to the Figures, the present invention is a system and method for an immersive light and sound experience. Now referring to FIG. 1, the physical components 100 and layout of one embodiment of the present invention is illustrated. The physical components 100 of the immersive light and sound show can be installed in an enclosed room, an open room/bar area, entryway, game rooms, restaurant, restaurant sections, customer gathering areas, or prize redemption areas in an arcade, casino, or similar establishments.

In alternative embodiments, the immersive experience taught by the present invention can be installed at Location Based Entertainment (LBE) and Family Entertainment Centers (FEC) in addition to the other types of locations it can be installed into as previously mentioned. The Immersive Experience can be independent and/or in conjunction with the locations current AV Hardware/Software system and/or their gaming card system.

The physical components shown in FIG. 1 are illustrative of one exemplary embodiment. In one such embodiment, a plurality of lights 102 and 106 and speakers 107 and 101 are generally located across a ceiling 110. Additionally, a scent dispenser 104 can be located on a ceiling 110 as well. A control panel 103 can be located or mounted on a wall 109 or at any other location as desired.

In FIG. 1, the exemplary embodiment is that of a birthday party 108, but the system and method can be adapted to other events and themes. In the exemplary birthday party 108 embodiment, a plurality of lights 102 and 106 and speakers 107 and 101 are located on the ceiling 110 will provide the audio and visual experience. The scent dispenser 104 is shown illustrating the scent of vanilla cake 105 to match the of the experience being a birthday party 108 and based on known properties of the vanilla scent, but any available scent such as cherry or pine, or any developed future scent could be selected for dispensing.

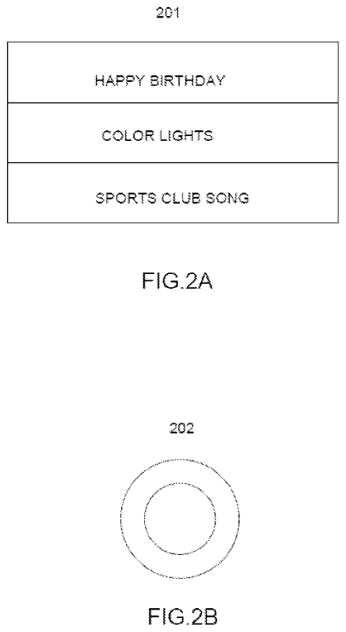

Now referring to FIGS. 2a and 2b, control panel 103 examples are illustrated. Referring to FIG. 2a, a control panel 103 can be comprised of a selectable menu via a touch panel 201 for selecting pre-programmed immersive experience programs such as “Happy Birthday”, “Color Lights”, “Sports Club Song”, and image mapping as desired for the immersive experience desired to be delivered.

In FIG. 2b, a typical on/off button 202 could be used as a remote control panel 103 or button 202 to simply turn on and off the experience, or to start a pre-programmed experience for execution/delivery to the room. In another embodiment the button 202 could be a multi-button with colored buttons for selecting different pre-programmed themes or immersive experience routines.

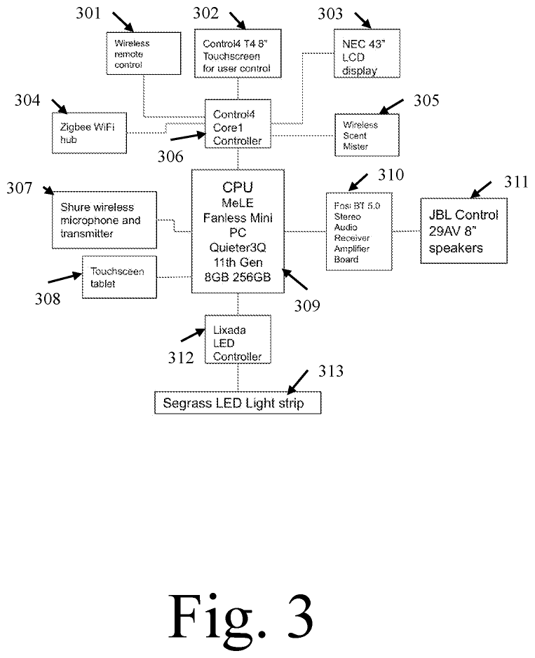

FIG. 3 is a flow chart illustrating the physical components executing and controlling the audio and visual components of the immersive experience.

In one embodiment as illustrated in FIG. 3, a microprocessor 309 and controller 306 are connected between the control panel 302 and physical components such as the lights 313, speakers 311, scent dispenser 305, etc. The microprocessor 309 is used for the storing in memory one or more pre-programmed immersive experiences and for controlling, presenting, and delivering a selected pre-programmed show to the room and participants for a fixed period of time.

Options stored in memory and selectable include, but are not limited to, selectable sound profiles for bass, the “Happy Birthday” song, and sport club song(s). In a single immersive experience, like for a birthday, a pre-programmed immersive experience might include a light show in combination with playing the “Happy Birthday” song.

In another immersive experience, one or more songs may be played in combination with a pre-programmed light show for a longer period of time or possibly on a loop depending on the immersive experience desired.

In other embodiments, the base components illustrated in FIG. 1 may be supplemented or replaced with laser lights, or rope LED lights 313, projection screens or projectors, pictures, video, and other sensory technologies for visual displays. With respect to sound, subwoofer(s), and/or a plug in for user or third/party music players can be included.

As illustrated in FIG. 3 for one exemplary embodiment, the microprocessor 309 is connected to a controller 306, stereo audio receiver amplifier board 310, LED or light controller 312, a touchscreen tablet 308, and a wireless microphone and transmitter 307.

The microprocessor 309 communicates with the led controller 312 which controls the lights 313 to deliver the lighting experienced of a selected pre-programmed show.

The microprocessor 309 can receive input from a touchscreen tablet 308 or from a wireless microphone and transmitter 307 to received input and feedback to control and/or adjust the pre-programmed light show based on the received input, such as including audio from the microphone 307 over the speakers 311 or adjustments or changes received from the tablet 308 which are then processed by the microprocessor 309 and disseminated to the corresponding hardware to make the input effective.

The microprocessor 309 communicates with the stereo audio receiver amplifier board 310 which controls the speakers 311 to deliver the sound/audio experienced of a selected pre-programmed show.

The microprocessor 309 communicates through the controller 306 with a touchscreen for user control 302 for receiving input.

The microprocessor 309 communicates through the controller 306 with a WIFI hub 304 for receiving input.

The microprocessor 309 communicates through the controller 306 with a wireless remote control 301 for receiving input.

The microprocessor 309 communicates through the controller 306 with a display 303 for displaying visual output.

The microprocessor 309 communicates through the controller 306 with a wireless scent mister 305 for scent output.

In an alternative embodiment the room or space can further include a wireless microphone 307 that can be either static or interactive with the song and/or lights. The system can also include/provide a plug in for third party devices and also provide wireless connection for third party devices. A feedback enabled microphone 307 for karaoke makes the environment dynamic instead of static or pre programmed.

In an alternative embodiment a digital or hard button can be used to start the show.

In still another alternative embodiment, the wireless microphone 307 and microprocessor 309 can communicate with a karaoke machine, device, or service to deliver a form of entertainment, offered typically by bars and clubs, in which people take turns to sing popular songs into a microphone 307 over prerecorded backing tracks over a web based platform like YOUTUBE. The wireless microphone and microprocessor communicate with a karaoke machine to combine the audio recorded by the microphone with the audio from the prerecorded backing tracks and displays or streams them over a web based platform.

Additionally, the present invention can be configured to provide automotive karaoke, where lighting, and projection can be added individually or in combination through the microprocessor and an additional karaoke controller to an automobile.

In another embodiment, the present invention can be combined with a debit card system trigger, from a game for high value winnings.

In other alternative embodiments, the present invention may be used in combination with specific locations and themes or room/location activities such as a jackpot for a casino, an arcade type game room, and in a bar experience to drive item sales or experience.

More specifically, in a casino, the present invention can be used in connection with a jackpot being hit to release a selected scent. Additionally in a casino, remote area displays can be used to display the jackpot, which can be a light or a video feed from the microprocessor 309.

In a bowling lane, the present invention can have a pre-programmed theme or immersive experience routine run in response to a strike.

In any location a strobe light or plurality of flashing lights near the entrance can be part of the light show to simulate a VIP entrance and emulate the or replicate a paparazzi or red carpet type experience.

In yet another alternative embodiment, the present invention can be used in combination with seat speakers for hearing impaired people to feel the bass or the audio. The present invention can incorporate any number of tactile response components.

In other alternative embodiments, the touchscreen control panel 302 can further be equipped with a software application or “APP” or additional communications with the controller 306 to add WIFI, BLUETOOTH, other wireless connectivity, or other remote activated experiences.

The present invention can also be included in the microprocessor 309 which is connected to an artificial intelligence (AI) module to add an interactive element to an immersive experience. In one embodiment, the microprocessor 309 can be connected to a third party AI application or software such as AMAZON ALEXA, GOOGLE ASSISTANT, or any other number of third party AI assistants which enabled one or more users to ask questions and get answers.

In another alternative AI embodiment, an AI assistant can be combined with a mirror or other static or interactive display of an image to create a party room attendant experience, similar to emulating or duplicating a magic mirror.

In a final alternative embodiment, the present invention can be white labeled for businesses. The microprocessor 309 would run/execute a white label software application or “APP” which would control the light and audio show to add commercials for revenue generation. Additionally, the white labeled “APP” could also control the scent for small business with related scent products.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A system for an immersive light experience comprising:
a plurality of physical components installed at a location;
a plurality of lights generally located across a ceiling;
a plurality of speakers generally located across a ceiling;
a scent dispense can be located on a ceiling as well;
a microprocessor;
a controller;
a stereo audio receiver amplifier board for controlling one or more speakers;
an LED or light controller for controlling one or more lights; and
a control panel located or mounted on a wall or at any other location as desired.

2. The system of claim 1, wherein
the location includes
an enclosed room,
an open room/bar area,
entryway, game rooms,
a restaurant,
restaurant sections,
customer gathering areas, or
prize redemption areas in an arcade, casino, or similar establishment.

3. The system of claim 1, wherein
the plurality of lights and speakers located on the ceiling provide the audio and visual experience.

4. The system of claim 1, wherein
the scent dispenser dispenses a scent to match that of the immersive experience.

5. The system of claim 4, wherein
the scent is any available scent such as vanilla, cherry, or pine, or any developed future scent could be selected for dispensing.

6. The system of claim 1, wherein
the control panel is comprised of a selectable menu via a touch panel or a wireless touchscreen tablet for selecting pre-programmed immersive experience programs as desired for the immersive experience desired to be delivered.

7. The system of claim 1, wherein
a typical on/off button is used as a remote control panel or button to simply turn on and off the experience, or to start a pre-programmed experience for execution/delivery to the room.

8. The system of claim 7, wherein
a digital or hard button is used to start the show; and
the button is a multi-button with colored buttons for selecting different pre-programmed themes or immersive experience routines.

9. The system of claim 1, further comprising
the microprocessor and controller connected between the control panel and physical components such as the lights, speakers, scent dispenser; and
the microprocessor and controller having a memory for the storing in memory one or more pre-programmed immersive experiences and for controlling, presenting, and delivering a selected pre-programmed show to the room and participants for a fixed period of time.

10. The system of claim 9, wherein
the microprocessor communicates with the led controller which controls the lights to deliver the lighting experienced of a selected pre-programmed show;
the microprocessor receives input from a touchscreen tablet or from a wireless microphone and transmitter to received input and feedback to control and/or adjust the pre-programmed light show based on the received input, including audio from the microphone over the speakers or adjustments or changes received from the tablet which are then processed by the microprocessor and disseminated to the corresponding hardware to make the input effective.

11. The system of claim 10, wherein
the microprocessor communicates with the stereo audio receiver amplifier board which controls the speakers to deliver the sound/audio experienced of a selected pre-programmed show;
the microprocessor communicates through the controller with a touchscreen for user control for receiving input;
the microprocessor communicates through the controller with a WIFI hub for receiving input;
the microprocessor communicates through the controller with a wireless remote control for receiving input;
the microprocessor communicates through the controller with a display for displaying visual output; and
the microprocessor communicates through the controller with a wireless scent mister for scent output.

12. The system of claim 9, wherein
options stored in memory and selectable include, but are not limited to, selectable sound profiles for bass, the “Happy Birthday” song, and sport club song(s).

13. The system of claim 9, wherein
in a single immersive experience, a pre-programmed immersive experience might include a light show in combination with playing a song.

14. The system of claim 9, wherein
in an immersive experience, one or more songs are played in combination with a pre-programmed light show for a period of time or possibly on a loop.

15. The system of claim 9, further comprising
supplemental or replacement lights including one or more
laser lights,
rope LED lights,
projection screens or projectors,
pictures,
video, and
other sensory technologies for visual displays.

16. The system of claim 9, further comprising
supplemental or replacement sound device including one or more
subwoofers, and
a plug in for user or third/party music players.

17. The system of claim 9, further comprising
a wireless microphone and transmitter which is either static or interactive with the song and/or lights;
the wireless microphone also provides feedback for karaoke and makes the environment dynamic instead of static or pre programmed; and
a plug in for third party devices provides wireless connection for third party devices.

18. The system of claim 17, further comprising
the wireless microphone and microprocessor communicate with a karaoke machine, device, or service to deliver a form of entertainment;
the wireless microphone and microprocessor communicate with a karaoke machine to combine the audio recorded by the microphone with the audio from the prerecorded backing tracks and displays or streams them over a web based platform; and
configured to provide automotive karaoke, where lighting, and projection can be added individually or in combination through the microprocessor and an additional karaoke controller to an automobile.

19. The system of claim 17, further comprising
a debit card system trigger, from a game for high value winnings.

20. The system of claim 17, wherein
when used in combination with a casino, arcade, or other gaming location, in connection with a jackpot being hit;
a scent dispenser releases a selected scent; and
remote area displays can be used to display the jackpot, which can be a light or a video feed from the microprocessor.

21. The system of claim 17, wherein
when used in combination with a bowling lane or alley;
a pre-programmed theme or immersive experience routine run in response to a strike.

22. The system of claim 17, further comprising
a strobe light or plurality of flashing lights near the entrance as part of the light show to simulate a VIP entrance and emulate the or replicate a paparazzi or red carpet type experience.

23. The system of claim 17, further comprising
seat speakers for hearing impaired people to feel the bass or the audio.

24. The system of claim 17, wherein
the touchscreen control panel is further equipped with a software application to add wireless connectivity or remote activated experiences.

25. The system of claim 17, wherein
the microprocessor is connected to an artificial intelligence (AI) module to add an interactive element to an immersive experience;
the microprocessor is connected to a third party AI application which enables one or more users to ask questions and get answers.

26. The system of claim 15, wherein
the AI module is combined with a mirror or other static or interactive display of an image to create a party room attendant experience.

27. The system of claim 15, wherein
the microprocessor runs/executes a white label software application to control the light and audio show to add commercials for revenue generation; and
the microprocessor runs/executes a white label software application controls the scent dispenser.
